(19) United States
(12) Patent Application Publication
(10) Pub. No.: US 2020/0030259 A1
(43) Pub. Date: Jan. 30, 2020

(54) Antiestrogens and/or Aromatase Inhibitors for Use in Treating Obesity and Related Symptoms

(71) Applicant: Repros Therapeutics Inc., The Woodlands, TX (US)

(72) Inventors: Joseph S. Podolski, The Woodlands, TX (US); Jaye Thompson, The Woodlands, TX (US); Martin Sandel, The Woodlands, TX (US)

(21) Appl. No.: 16/335,693

(22) PCT Filed: Sep. 25, 2017
(86) PCT No.: PCT/US2017/053234

§ 371 Date: Mar. 22, 2019

Related U.S. Application Data
(60) Provisional application No. 62/399,933, filed on Sep. 26, 2016.

(51) Int. Cl. A61K 31/135; A61K 9/00; A61K 31/4196; A61K 31/566; A61K 31/437; A61K 31/45; A61K 31/585; A61P 3/04

(52) U.S. Cl. CPC A61K 31/135; A61K 9/0053; A61K 31/4196; A61P 3/04; A61K 31/437; A61K 31/45; A61K 31/585; A61K 31/566

(57) ABSTRACT

The present invention relates to the use of an antiestrogen such as trans-clomiphene or an analogue or salt thereof, and/or an aromatase inhibitor to treat obesity and/or a symptom associated therewith.

### FIELD OF THE INVENTION

The present invention relates to compositions and methods for treating obesity and related symptoms by administering to a subject in need thereof an effective amount of an antiestrogen such as a selective estrogen receptor modulator, and/or an aromatase inhibitor. Also provided is a method for losing weight by administering to an obese male an effective amount of an antiestrogen and/or an aromatase inhibitor.

### SUMMARY

In several embodiments, the present invention is directed to methods for treating obesity by administering to a human subject in need thereof an effective amount of an antiestrogen and/or aromatase inhibitor.

In related embodiments, the present invention is directed to methods for preventing and/or treating one or more symptoms associated with obesity by administering to a human subject in need thereof an effective amount of an antiestrogen and/or aromatase inhibitor. In a related embodiment, the present invention provides a method for increasing lean muscle mass in a human subject by administering to the subject an effective amount of an antiestrogen and/or aromatase inhibitor.

In other embodiments, the present invention is directed to a method for reducing weight in a human subject by administering to the subject an effective amount of an antiestrogen and/or aromatase inhibitor.

The human subject to be treated according to the herein described methods may be a male or female in need thereof. In preferred embodiments, the subject is a human male with low (e.g. below about 300 ng/dl) or low-normal (e.g. below about 400 ng/dl) serum testosterone levels and a body mass index (BMI) ≥30. In a related embodiment, the subject is a secondary hypogonadal human male with a body mass index (BMI) ≥30, a secondary hypogonadal human male with a BMI ≥35 a secondary hypogonadal male with a BMI ≥40 or a secondary hypogonadal male with a BMI any range therebetween (e.g. BMI between 35 and 40, inclusive). In a particularly preferred embodiment, the subject is a secondary hypogonadal human male with a BMI ≥30 and is less than 60 years of age (e.g. is between 40 and 59 years of age).

In some preferred embodiments, the subject is a human male less than 60 years of age with low or low-normal serum testosterone levels and a testosterone:estradiol (T:E) ratio of below about 10, below about 9, below about 8, below about 7, below about 6 or below about 5, or from about 4 to about 5 or from about 4 to about 6 or from about 3 to about 7, or from about 4 to about 7 or from about 4 to about 8 or from about 5 to about 8 or from about 4 to about 10.

In related embodiments, the subject is a human male less than 60 years of age with low or low-normal serum testosterone levels and an estradiol level of at least 20 μg/ml, at least 25 μg/ml, at least 30 μg/ml, at least 35 μg/ml, at least 40 μg/ml, at least 50 μg/ml, between 30 and 50 μg/ml, between 30 and 60 μg/ml or any range therebetween.

In some embodiments, the subject to be treated according to the herein described methods is administered an antiestrogen. In preferred embodiments the antiestrogen is a selective estrogen receptor modulator (SERM) such as trans-clomiphene or a salt or analogue thereof. In some aspects, the antiestrogen is administered in a composition comprising a pharmaceutically acceptable carrier, excipient or diluent. In some aspects, the treatment method does not comprise co-administering an aromatase inhibitor to the subject.

In other embodiments, the subject to be treated according to the herein described methods is administered an aromatase inhibitor. In some aspects, the aromatase inhibitor is administered in a composition comprising a pharmaceutically acceptable carrier, excipient or diluent. In some aspects, the treatment method does not comprise co-administering an antiestrogen to the subject.

In some embodiments, the subject to be treated according to the herein described methods is co-administered an antiestrogen and an aromatase inhibitor. In some aspects, the antiestrogen and aromatase inhibitor are administered sequentially in which case a first dose of the antiestrogen may be administered prior to a first dose of aromatase inhibitor or vice versa and may include a phase where treatment with the antiestrogen and treatment with the aromatase inhibitor overlap. In some aspects, the antiestrogen and aromatase inhibitor are administered simultaneously to the subject. In related aspects, the subject is administered a composition comprising an antiestrogen, an aromatase inhibitor and a pharmaceutically acceptable carrier, excipient or diluent. In preferred embodiments, an antiestrogen and aromatase inhibitor are administered within 12, 24, 48, or 72 hours or within 4, 5, 6 or 7 days, or within 8, 9, 10, 11, 12, 13, 14, 15, 16, 17, 18, 19, 20, 21, 22, 23, 24, 25, 26, 27, 28, 29, 30 or 31 days of each other.

In some embodiments, the herein described treatment methods further comprise a prescribed (e.g. physician-prescribed) weight reduction regimen. The prescribed weight reduction regimen comprises a prescribed exercise regimen and optionally a prescribed diet. In some aspects, the duration of the prescribed weight reduction regimen is co-extensive with the duration of treatment with the antiestrogen and/or aromatase inhibitor. In related aspects, the prescribed weight reduction regimen is initiated at about the first dose of antiestrogen and/or aromatase inhibitor and the duration of the weight reduction regimen exceeds the duration of treatment with the antiestrogen and/or aromatase inhibitor.

The antiestrogen and/or aromatase inhibitor is administered to the subject for a period of time sufficient to achieve the desired result. In some aspects, an effective amount of antiestrogen is administered daily or every other day to a human subject for a period of two, three, four five or 6 weeks, at least 3 months, at least 6 months, at least one year or even at least 2, 3, 4 or 5 years in order to treat obesity or a symptom associated therewith or to reduce weight in the subject. In some preferred embodiments, a human subject is administered an effective dose of the antiestrogen every day or every other day by the oral route.

In some embodiments, the present invention is directed to a method for reversing secondary hypogonadism in a human male with secondary hypogonadism and a BMI ≥30 (e.g. BMI ≥35) who is less than 60 years of age (e.g. between 40 and 59 years of age) by administering an antiestrogen and/or aromatase inhibitor for a period of at least about six months (e.g. about six months, about a year, about 6 months to a year, etc.) in combination with a prescribed weight loss regimen. Preferably, LH and testosterone levels in the subject remain in the normal range for a period of at least 6 months, at least 1 year, at least 2 years, at least 3 years, at least 4 years or at least 5 years after cessation of treatment with the antiestrogen and/or aromatase inhibitor.

Also provided is a pharmaceutical composition that comprises an antiestrogen and/or an aromatase inhibitor or and a pharmaceutically acceptable carrier, excipient or diluent, for use in the treatment of obesity or a symptom associate therewith or for reducing weight in a subject. In some aspects, the composition is formulated for oral use.

In preferred embodiments, a subject is administered an amount of trans-clomiphene or a salt or analogue thereof, preferably substantially free of cis-clomiphene, effective to treat obesity or a symptom associated therewith or to reduce weight in the subject. In some embodiments, the analogue of trans-clomiphene is a metabolite of trans-clomiphene such as (E)-4-OH-Clomiphene), (E)-4-OH-desethyl Clomiphene or (E)-4,4′-di-OH Clomiphene.

Preferred dosages of trans-clomiphene or an analogue or salt thereof include 25 mg to 100 mg, 25 mg to 50 mg, 12.5 mg to 100 mg, 12.5 mg to 50 mg, 12.5 mg to 25 mg, 5 mg to 20 mg, and 5 mg to 15 mg trans-clomiphene or an analogue or salt thereof. A particularly preferred dosage is 25 mg/day by oral route. Preferably trans-clomiphene is administered daily or every other day, but may also be administered periodically such as weekly, bi-weekly or even monthly. Preferably periodic administration of trans-clomiphene is preceded by daily administration for a period of time sufficient to achieve therapeutic levels of testosterone.

### BRIEF DESCRIPTION OF THE FIGURES

FIGS. 1A and 1B illustrate body mass index (BMI) and morning testosterone levels respectively, at baseline and following 3 months and 6 months of daily administration of 12. 5 mg or 25 mg enclomiphene or placebo treatment in combination with a weight loss regimen in obese middle aged human males with secondary hypogonadism.

FIGS. 2A and 2B illustrate lean mass (as a percentage of total mass in kilograms, measured by dual energy X-ray Absorptiometry (DXA)) and total testosterone levels at baseline and at the 3 month and 6 month time points in the subjects described at FIGS. 1A and 1B.

FIG. 3 illustrates percent improvement from baseline total score at the 3 month and 6 month time points in subjects described at FIGS. 1A and 1B (n=15 per group) in the DISF-SR (Derogatis Interview for Sexual Functioning), IWQOL (Impact of Weight on Quality of Life questionnaire) and SF-36 (Medical Outcomes Study Short Form).

### DETAILED DESCRIPTION

A “therapeutically effective amount” of a drug is an amount effective to demonstrate a desired activity of the drug. A therapeutically effective amount of antiestrogen or aromatase inhibitor may be an amount effective to achieve a reduction in weight in a subject or to treat obesity and/or to ameliorate, i.e., noticeably reduce, one or more of the symptoms of obesity in a subject.

Symptoms associated with obesity include, without limitation, fatigue (or lack of energy), depression, irritability, lack of focus, poor libido muscle weakness. In some preferred embodiments, the subject is administered an amount of an antiestrogen (e.g. trans-clomiphene) and/or aromatase inhibitor effective to improve one or more of these symptoms as measured by an improvement in the DISF-SR (Derogatis Interview for Sexual Functioning), IWQOL (Impact of Weight on Quality of Life questionnaire) and/or SF-36 (Medical Outcomes Study Short Form) total score by at least 30, at least 35, at least 40, at least 45, at least 50, at least 55, or at least 60 percent compared to baseline (pre-treatment) total score(s).

By “antiestrogen” it is meant a compound that prevents estrogens from expressing their effects on estrogen dependent target tissues consequently antagonizing a variety of estrogen-dependent processes. In all cases, antiestrogens useful in the practice of the instant invention are those capable of blocking the negative feedback exerted by normal estrogens on the pituitary leading to increases in LH and FSH. In men, these increased levels of gonadotropins stimulate the Leydig cells of the testes and result in the production of higher testosterone levels. Antiestrogens useful in the practice of the instant invention may be pure antiestrogens or may have partial estrogenic action as in the case of the selective estrogen receptor modulators (SERMs) which exhibit antiestrogenic properties in some tissues and estrogenic tissues in others. Trans-clomiphene is a preferred antiestrogen for use in the methods described herein, preferably in a form substantially free of cis-clomiphene (e.g. in a composition comprising about 100% w/w trans-clomiphene and about 0% w/w cis-clomiphene as active agent).

Pure antiestrogens of the invention include, without limitation: RU 58,688, described in Van de Velde et al, Ann. NY Acad. Sci., 761(3): 164-175 (1995); 13-methyl-7-[9-(4,4,5,5,5-pentafluoropentylsulfinyl)nonyl]-7,8,9, 11, 12, 13, 14, 15, 16,17-decahydro-6H-cyclopenta[a]-phenanthrene-3,17-diol (ICI 182,780/fulvestrant) and other compounds described in EP 0138504; N-butyl-11-[(7R,8S,9S,13S,14S,17S)-3,17-dihydroxy-13-methyl-6,7,8,9,11,12,14,15,16, 17-decahydrocyclopena[a]phenanthren-7-yl]-N-methyl-undecanamide (ICI 164,384), described in Wakeling and Bowler, J. Endocrin., 112:R7-R110 (1987); (#)-7-pivaloyloxy-3-(4′pivaloyloxyphenyl)-4-methyl-2-(4″ (2″piperidinoethoxy)phenyl)-2H-benzopyran (EM-800/SCH 57050) and other compounds described in WO 96/26201; (2S)-3-(4-hydroxyphenyl)-4-methyl-2-[4-[2-(1-piperidyl)ethoxy]phenyl]-2H-chromen-7-ol (EM-652/SCH 57068) and the like.

SERMs useful in the methods of the invention include, without limitation, triphenylalkylenes such as triphenylethylenes, which include: 2-[4-(1,2-diphenylbut-1-enyl)phenoxy]-N,N-dimethyl-ethanamine (tamoxifen) and other compounds described in U.S. Pat. No. 4,536,516, incorporated herein by reference; Trans-4-(1-(4-(2-dimethylamino)ethoxy)phenyl)-2-phenyl-1-butenyl)phenol (4-hydroxytamoxifen) and other compounds described in U.S. Pat. No. 4,623,660, incorporated herein by reference; 1-[4′-dimethylaminoethoxy)phenyl]-1-(3′-hydroxyphenyl)-2-phenylbut-1-ene (droloxifene) and other compounds described in U.S. Pat. No. 5,047,431, incorporated herein by reference; 2-[p-[(Z)-4-chloro-1,2-diphenyl-1-butenyl]phenoxy]-N,N-dimethylethylamine (toremifene) and other compounds described in U.S. Pat. Nos. 4,696,949, 5,491,173 and 4,996,225, each of which is incorporated herein by reference; (E)-1-(2-(4-(I-(4-iodo-phenyl)-2-phenyl-but-1-enyl)-phenoxy)-ethyl)-pyrrol idinone (idoxifene) and other compounds described in U.S. Pat. No. 4,839,155, incorporated herein by reference; clomiphene and both its isomers; and compounds described in U.S. Pat. Nos. 4,696,949 and 5,491,173 and 6,576,645, each of which is incorporated herein by reference.

SERMS useful in the methods of the invention also include, without limitation, benzothiphene derivatives such as: [6-hydroxy-2-(4-hydroxyphenyl)-benzothiophen-3-yl]-[4-[2-(1-piperidinyl)ethoxy)phenyl]-methanone (raloxifene) and other compounds described in U.S. Pat. Nos. 4,418,068 and 5,393,763, both of which are incorporated herein by reference; LY353381; and LY335563 and other compounds described in WO 98/45286, WO 98/45287 and WO 98/45288; benzopyran derivatives such as: (#)-7-pivaloyloxy-3-(4 ′pivaloyloxyphenyl)-4-methyl-2-(4″-(2″piperidinoethoxy)phenyl)-2H-benzopyran (EM 800/SCH 57050) and other compounds described in WO 96/26201; (25)-3-(4-hydroxyphenyl)-4-methyl-2-[4-[2-(1-piperidyl)ethoxy]phenyl]-2H-chromen-7-ol (EM 652); naphthalene derivatives such as: Cis-6-phenyl-5-[4-(2-pyrrolidin-1-yl-ethoxy)-phenyl]-5,6,7,8-tetrahydronaphthalen-2-ol (lasofoxifene/CP 336,156) and other compounds described in U.S. Pat. No. 5,552,412; 3,4-dihydro-2-(p-methoxyphenyl)-1-naphthyl-p-[2-(1-pyrrolidinyl)ethoxy]phenyl ketone (trioxifene/LY133314) and other compounds described in U.S. Pat. No. 4,230,862, incorporated herein by reference; and 1-(4-Substituted alkoxy)benzyl)naphthalene compounds such as those described in U.S. Pat. No. 6,509,356, incorporated herein by reference; chromans such as 3,4-trans-2,2-dimethyl-3-phenyl-4-[4-(2-(2-(pyrrolidin-1-yl)ethoxy)phenyl]-7-methoxychroman (levormeloxifene) and other compounds described in WO 97/25034, WO 97/25035, WO 97/25037 and WO 97/25038; and 1-(2-((4-(-methoxy-2,2, dimethyl-3-phenyl-chroman-4-yl)-phenoxy)-ethyl)-pyrrolidine (centchroman) and other compounds described in U.S. Pat. No. 3,822,287, incorporated herein by reference.

Other SERMs of the invention include, without limitation, the compounds described in U.S. Pat. Nos. 6,387,920, 6,743,815, 6,750,213, 6,869,969, 6,927,224, 7,045,540, 7,138,426, 7,151,196, and 7,157,604, each of which is incorporated herein by reference.

Further non-limiting antiestrogens of the invention include: 6α-chloro-16α-methyl-pregn-4-ene-3,20-dione (clometherone); 6-chloro-17-hydroxypregna-1,4,6-triene-3,20-dione (delmadinone); 1-[2-[4-[1-(4-methoxyphenyl)-2-nitro-2-phenylethenyl]phenoxy]ethyl]-pyrrolidine (nitromifene/CN-55,945-27); and 1-[2-[p-(3,4-Dihydro-6-methoxy-2-phenyl-1-naphthyl)phenoxy] ethyljpyrrolidine (nafoxidene).

Further non-limiting antiestrogens of the invention include indoles such as those disclosed in J. Med. Chem., 33:2635-2640 (1990), J. Med. Chem., 30:131-136 (1987), WO 93/10741, WO 95/17383, WO 93/23374 and U.S. Pat. Nos. 6,503,938 and 6,069,153, both of which are incorporated herein by reference.

Further non-limiting antiestrogens of the invention include 2-[3-(1-cyano-1-methyl-ethyl)-5-(1H-1,2,4-triazol-1-ylmethyl)phenyl]-2-methyl-propanenitrile (anastrozole) and other compounds described in EP 0296749; 6-Methylenandrosta-1,4-diene-3,17-dione (exemestane) and other compounds described in U.S. Pat. No. 4,808,616, incorporated herein by reference; 4-[(4-cyanophenyl)-(1,2,4-triazol-1-yl)methyl]benzonitrile (letrozole) and other compounds described in U.S. Pat. No. 5,473,078, incorporated herein by reference; 1-[4′-dimethylaminoethoxy)phenyl]-1-(3′-hydroxyphenyl)-2-phenylbut-1-ene (droloxifene) and other compounds described in U.S. Pat. No. 5,047,431, incorporated herein by reference; 2α,3α-Epithio-5α-androstan-17β-ol (epitiostanol); 2α,3α-Epitio-5α-androstane-17β-yl-1-methoxycyclopentyloxy (mepitiostane); 4-[(2Z,4Z)-4-(4-hydroxyphenyl)hexa-2,4-dien-3-yl]phenol (cycladiene) and other compounds described in U.S. Pat. Nos. 2,464,203 and 2,465,505, both of which are incorporated herein by reference; CI-680 described in Unlisted Drugs, 28(10): 169(0) (1976); CI-628 described in Unlisted Drugs, 26(7): 106(1) (1974); 13-ethyl-17α-ethynl-17β-hydroxygona-4,9,1-trien-3-one (R2323); diphenol hydrochrysene and erythyro-MEA both described in Geynet, et ah, Gynecol. Invest. 3(1):2-29 (1972); 1-[1-chloro-2,2-bis(4-methoxyphenyl)ethenyl]-4-methoxy-benzene (chlorotrianisene) described in Merck Index, 101 ed., #2149; 1-[4-(2-Diethylaminoethoxy)phenyl]-1-phenyl-2-(p-anisyl)ethanol (ethamoxytriphetol) described in Merck Index, 101 ed., #3668; and 2-p-Chlorophenyl-[p-(2-diethylaminoethoxy)phenyl]-1-p-tolylethanol (triparanol) and other compounds described in U.S. Pat. No. 2,914,562, incorporated herein by reference.

Still other antiestrogens of the invention include, without limitation: (2e)-3-(4-((le)-1,2-diphenylbut-1-enyl)phenyl)acrylic acid (GW5638), GW7604 and other compounds described in Wilson et al, Endocrinology, 138(9):3901-3911 (1997) and WO 95/10513; 1-[4-(2-diethylaminoethoxy)phenyl]-2-(4-methoxyphenyl)-1-phenyl-ethanol (MER-25), N,N-diethyl-2-[4-(5-methoxy-2-phenyl-3H-inden-1-yl)phenoxy]ethanamine hydrochloride (U-11,555A), 1-[2-[4-(6-methoxy-2-phenyl-3,4-dihydronaphthalen-1-yl)phenoxy]ethyl]pyrrolidine hydrochloride (U-Il, 100A), ICI-46,669, 2-[4-[(Z)-1,2-diphenylbut-1-enyl]phenoxy]-N,N-dimethyl-ethanamine; 2-hydroxypropane-1,2,3-tricarboxylic acid (ICI-46,474) and other compounds described in Terenius et al., Gynec. Invest., 3:96-107 (1972); 2-Hydroxy-6-naphthalenepropionic acid (allenolic acid); [4-[(4-acetyloxyphenyl)-cyclohexylidene-methyl]phenyl]acetate (cyclofenyl/ICl-48213); [6-hydroxy-2-(4-hydroxyphenyl)benzothiophen-3-yl]-[4-[(4-piperidyl)ethoxy]phenyl]methanone (keoxifene); 4-[(Z)-1-[4-(2-dimethylaminoethoxy)phenyl]-2-(4-propan-2-ylphenyl)but-1-enyl]phenol (DP-TAT-59/miproxifene); (1RS,2RS)-4,4 ‘-diacetoxy-5,5’-difluoro-(1-ethyl-2-methylene)di-m-phenylenediacetate (acefluranol); 6-hydroxy-2-(p-hydroxyphenyl)-benzo(b)thien-3-yl[2-(1-pyrrolidinyl)-ethoxyphenyl]ketone (LY-117018); and [6-hydroxy-2-(4-hydroxy-phenyl)benzo(b)thien-3-yl]-[4-(2-(1-piperdinyl)-ethoxy)phenyl]methanone (LY-156758).

Still other antiestrogens of the invention include, without limitation: non-steroidal estrogen receptor ligands such as those described in U.S. Pat. Nos. 5,681,835, 5,877,219, 6,207,716, 6,340,774 and 6,599,921, each of which is incorporated herein by reference; steroid derivatives such as those described in U.S. Pat. No. 4,659,516, incorporated herein by reference; 7α-11-aminoalkyl-estratrienes such as those described in WO 98/07740; 11-β-halogen-7α-substituted estratrienes such as those described in WO 99/33855; 17α-alkyl-17β-oxy-estratrienes such as those described in U.S. patent application Ser. No. 10/305,418, incorporated herein by reference; 2-phenyl-1-[4-(2-aminoethoxy)-benzyl]-indoles such as those described in U.S. Pat. No. 7,132,417, incorporated herein by reference; 4-fluoroalkyl-2h-benzopryans such as those described in U.S. Pat. No. 6,844,336, incorporated herein by reference; (4-(2-(2-aza-bicyclo[2.2.1]hept-2-yl)-ethoxy)-phenyl)-(6-hydroxy-2-(4-hydroxy-phenyl)-benzo[b]thiophen-3-yl)-methanone and other benzothiophenes described in WO 95/10513 and U.S. Pat. No. 4,133,814, incorporated herein by reference; 2-phenyl-1-[4-(2-aminoethoxy)-benzyl]-indoles such as those described in U.S. Pat. No. 5,998,402, incorporated herein by reference; 3-[4-(2-Phenyl-Indole-1-ylmethyl) Phenyl]—Acrylamides and other compounds described in U.S. Pat. No. 5,985,910, incorporated herein by reference; 2-phenyl-1-[4-(amino-1-yl-alk-1-ynyl)-benzyl]-1H-indol-5-ols and other compounds described in U.S. Pat. Nos. 5,780,497 and 5,880,137, both of which are incorporated herein by reference; steroids such as those described in U.S. Pat. Nos. 6,455,517, 6,548,491, 6,747,018 and 7,041,839, each of which is incorporated herein by reference; Di-(3′-hydroxyphenyl)-alkane compounds such as those described in U.S. Pat. No. 4,094,994, incorporated herein by reference; phenol derivatives such as those described in U.S. Pat. No. 4,751,240, incorporated herein by reference; 2,3-diaryl-2H-1-benzopyran analogs such as those described in Saeed et al., J. Med. Chem., 33:3210-3216 (1990) and Sharma et al, J. Med. Chem. 33:3216-3229 (1990); and benzofuran and triarylfuran analogs such as those described in Durani et al, J. Med. Chem., 32: 1700-1707 (1989)

The family of triphenylalkylene derivatives representing analogs of clomiphene is defined here to include all unmodified trans forms, as well as each of the 4-hydroxylated, the N-dealkylated and the 4-hydroxy-N-dealkylated analogs of trans-clomiphene, as well as all other molecules with substantially similar structures. Analogs of trans-clomiphene such as those described in Ernst, et al., J. Pharmaceut. Sci. 65:148 (1976) and the metabolites described herein are also useful in the practice of the present invention.

In a preferred embodiment, the antiestrogen trans-clomphene or a salt thereof such as trans-clomiphene citrate is administered to a subject to treat obesity or a symptom associated therewith or to reduce weight in the subject at a dose which may range from 1 to 200 mg or from 5 to 100 mg, preferably administered daily or every other day. The dosage of trans-clomphene may also be from 5 to 10 mg, from 5 to 12.5 mg, from 5 to 15 mg, from 5 to 20 mg, from 10 to 15 mg, from 10 to 20 mg, from 12.5 to 25 mg, from 12.5 to 50 mg, or from 25 mg to 50 mg. The dosage of trans-clomphene may also be 12.5 mg, 25 mg or 50 mg. A preferred dosage of trans-clomiphene is 25 mg per day.

In one embodiment, compositions of the invention comprise one or more pharmaceutically acceptable salts of trans-clomiphene or an analogue thereof. Depending on the process conditions the salt compound obtained may be either in neutral or salt form. Salt forms include hydrates and other solvates and also crystalline polymorphs. Both the free base and the salts of these end products may be used in accordance with the invention. A preferred salt is the citrate salt.

By “aromatase inhibitor” it is meant non-steroidal and steroidal compounds that inhibit the enzyme aromatase thereby preventing the conversion of androgens to estrogens, preferably those which inhibit aromatase activity in vitro with an IC₅₀ value of less than 10⁻⁵ M as well as their pharmaceutically acceptable salts. Preferred aromatase inhibitors for use in the methods herein described include without limitation anastrozole, letrozole, exemestane, vorozole, formestane, fadrozole, aminoglutethimide, testolactone, 4-hydroxyandrostenedione, 1,4,6-androstatrien-3,17-dione and 4-androstene-3,6,17-trione.

Antiestrogens and aromatase inhibitors for use in the methods described herein may be formulated, together with a pharmaceutically acceptable carrier, into pharmaceutical compositions which can be administered by any acceptable delivery method. In some embodiments, antiestrogens and/or aromatase inhibitors are formulated into solid dosage units such as tablets, (e.g. suspension tablets, bite suspension tablets, rapid dispersion tablets, chewable tablets, effervescent tablets, bilayer tablets, etc.), caplets, capsules (e.g., a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for oral administration. A preferable dosage form is a soft or hard gelatin capsule. Another preferable dosage form is a tablet. The compositions may also be in the form of sterile injectable solutions or emulsions for parenteral (including intravenous, intraarterial, intraperitoneal, subcutaneous, intramuscular, intrathecal and intraarticular) use. Such pharmaceutical compositions and unit dosage forms thereof may comprise ingredients in conventional proportions.

The term “oral administration” herein includes any form of delivery of a therapeutic agent or a composition thereof to a subject wherein the agent or composition is placed in the mouth of the subject, whether or not the agent or composition is swallowed. Thus, “oral administration” includes buccal and sublingual as well as esophageal (e.g. inhalation) administration

Acid addition salts may be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs may be used in accordance with the invention. A preferred salt is the citrate salt.

Base addition salts may also be used in accordance with the invention and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like.

Compositions of the invention can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition. Excipients include, by way of illustration and not limitation, diluents, disintegrants, binding agents, adhesives, wetting agents, lubricants, glidants, surface modifying agents or surfactants, fragrances, suspending agents, emulsifying agents, nonaqueous vehicles, preservatives, antioxidants, adhesives, agents to adjust pH and osmolarity (e.g. buffering agents), preservatives, thickening agents, sweetening agents, flavoring agents, taste masking agents, colorants or dyes, penetration enhancers and substances added to improve appearance of the composition.

Excipients optionally employed in compositions of the invention can be solids, semi-solids, liquids or combinations thereof. Compositions of the invention containing excipients can be prepared by any known technique of pharmacy that comprises mixing an excipient with a drug or therapeutic agent.

A therapeutically effective amount of the composition required for use in therapy varies with the length of time that activity is desired, and the age and the condition of the patient to be treated, among other factors, and is ultimately determined by the attendant physician. In general, however, doses employed for human treatment typically are in the range of about 0.001 mg/kg to about 500 mg/kg per day, for example about 1 μg/kg to about 1 mg/kg per day or about 1 μg/kg to about 100 μg/kg per day. For most large mammals, the total daily dosage is from about 1 to 100 mg, preferably from about 2 to 80 mg. The dosage regimen may be adjusted to provide the optimal therapeutic response. The desired dose may be conveniently administered in a single dose, or as multiple doses administered at appropriate intervals, for example as two, three, four or more subdoses per day.

Illustratively, a composition of the invention may be administered to a subject to provide the subject with an antiestrogen in an amount of about 1 μg/kg to about 1 mg/kg body weight, for example about 1 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg or about 1 mg/kg body weight.

In a preferred embodiment, compositions according to the present invention comprise trans-clomiphene at a dosage between one mg to about 200 mg (although the determination of optimal dosages is with the level of ordinary skill in the art). The composition may comprise trans-clomiphene at a dosage of about 1 mg, 2 mg, 3 mg, 4 mg, 5 mg, 10 mg, 12 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg or there between. The composition is preferably substantially free of cis-clomiphene and may comprise 0% w/w cis-clomiphene. Analogs of the trans-isomer of clomiphene such as those described in Ernst, et al. supra are also useful in the practice of the present invention.

All of the references referred to herein are incorporated by reference in their entirety.

The following Examples are meant to be illustrative of the invention and are not intended to limit the scope of the invention as set out is the appended claims.

### Example 1

### Phase 2 Clinical Trial

A randomized double blind placebo-controlled Phase 2 clinical study (duration=6 months) was initiated to study the effects of trans-clomiphene on testosterone, metabolic function, weight, body composition, measures of strength and quality of life assessments (made throughout the study) in obese men with acquired hypogonadotropic hypogonadism.

50 adult male subjects are enrolled in the study with an average age of 43.3 (stdev 9.2), average BMI of 36.8 (stdev 3.2), average waist size of 46.9″ (stdev 4.1) and average body fat percentage of 38.1 (stdev 5.2). Average baseline testosterone levels in the subjects was 221.9 ng/dl (stdev 52.7), average estradiol level in the subjects was 48.1 μg/ml (stdev 14.8) and the average T:E ratio was 4.95 (stdev 1.7; normal range of T:E is 20-25). The top four reported baseline symptoms (by % of enrollees) were (i) fatigue/lack of energy (96%) (ii) depression, irritability, lack of focus (74%) (iii) poor libido (60%) and (iv) muscle weakness (48%). The subjects were provided a low calorie commercial diet for 6 months, gym membership for 15 month and access to a personal trainer for 6 months. Subjects were randomized to receive treatment with 12.5 mg enclomiphene/day, 25 mg enclomiphene/day or placebo via oral capsule over a three month period.

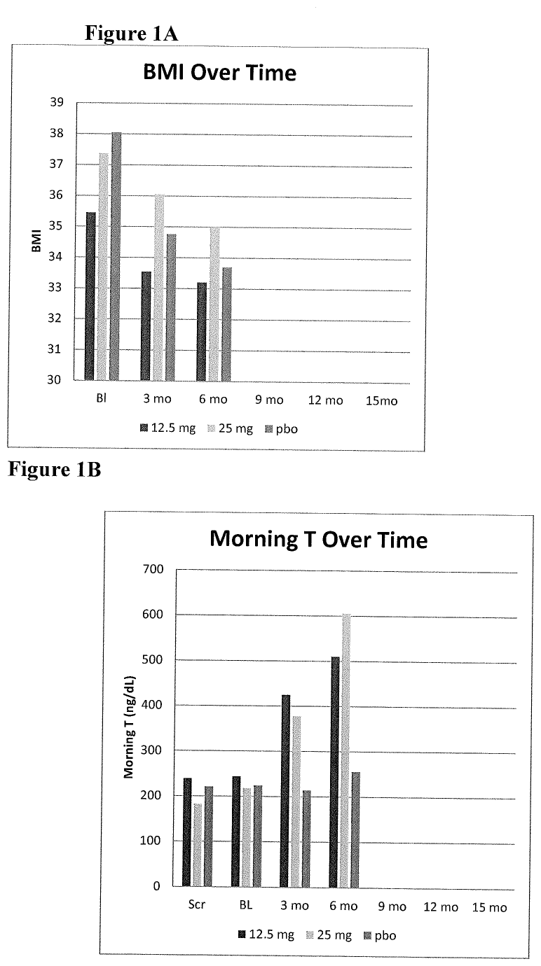

Interim six-month data are shown at FIGS. 1-3.

FIG. 1B demonstrates an increase in morning testosterone from ˜235 ng/dl at baseline to ˜505 ng/dl at six months in the 12.5 mg enclomiphene arm and from ˜285 ng/dl at baseline to ˜605 ng/dl at six months in the 25 mg enclomiphene arm. The difference was highly statistically significant (p<0.0001). In contrast, only an insignificant increase in morning testosterone levels were observed in the placebo group.

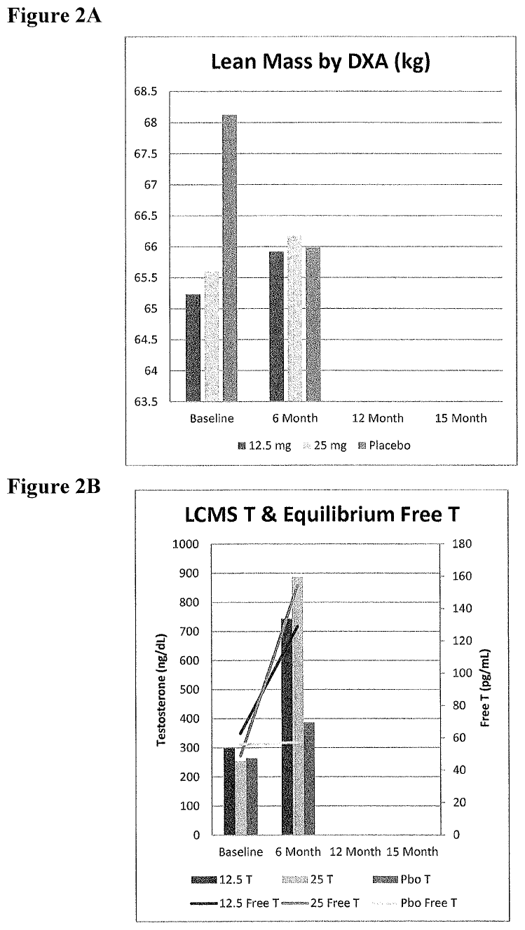

FIG. 2A demonstrates that at the six month time period, the placebo arm lost lean mass whereas in contrast 12.5 mg and 25 mg enclomiphene arms gained lean mass.

FIG. 2B demonstrates increases in free and total (LCMS) testosterone from baseline levels to the six month time point in enclomiphene and placebo arms.

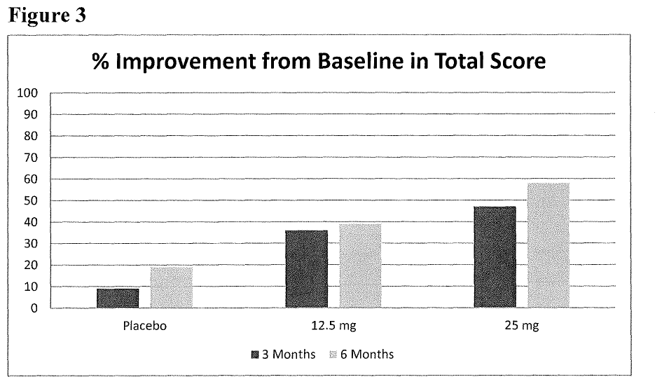

FIG. 3 demonstrates percent improvement relative to baseline in total score from the DISF-SR (Derogatis Interview for Sexual Functioning), IWQOL (Impact of Weight on Quality of Life questionnaire) and SF-36 (Medical Outcomes Study Short Form) in the enclomiphene and placebo arms. All improvements were statistically significant from baseline.

### Discussion

Together, these results indicate that antiestrogens such as trans-clomiphene, which effectively increase LH and FSH levels, will provide an effective treatment of obesity (concomitant with an increase in lean mass percentage) as well as an effective treatment of symptoms associated with obesity, particularly in human males with secondary hypogonadism that are under the age of 60 with BMI of ≥35 and estradiol levels exceeding 40 μg/ml.

### Example 2

### Treating Obesity with an Antiestrogen

Subjects in need of treatment for obesity are administered an amount of an antiestrogen (e.g. trans-clomiphene or a salt or analogue thereof) in an amount effective to increase LH and FSH to therapeutic levels over a treatment period of sufficient duration to achieve the desired result, preferably for a period of at least 3 months during which an effective dose is administered daily or every other day. FSH and LH levels may be assessed prior to initiating treatment and then monitored through the course of treatment to ensure that therapeutic levels of the pituitary hormones are reached. By way of nonlimiting example, a subject with obesity is administered 5 mg to 100 mg trans-clomiphene daily or every other day for a treatment period of at least three months, preferably at least six months. A reduction in one or more symptoms of obesity is observed during the treatment.

1. A method for treating obesity or a symptom associated therewith comprising administering an effective amount of an antiestrogen and/or aromatase inhibitor to a human male less than 60 years of age with secondary hypogonadism, and a body mass index ≥30.

2. The method of claim 1, wherein the human male is administered an antiestrogen and is not administered an aromatase inhibitor.

3. The method of claim 1, wherein an antiestrogen and an aromatase inhibitor are co-administered to the human male

4. The method of claim 1, wherein the human male is administered trans-clomiphene or an analogue or pharmaceutically acceptable salt thereof in an amount of from about 5 mg to about 100 mg, the composition being substantially free of cis-clomiphene.

5. The method of claim 4 wherein the human is administered from 5 mg to about 100 mg trans-clomiphene per day or every other day for at least 3 months or at least 6 months.

6. The method of claim 1, wherein the human is administered an amount of trans-clomiphene or an analogue or salt thereof effective to increase lean mass in the human male compared to pre-treatment (baseline) levels.

7. The method of claim 1, wherein the human is administered trans-clomiphene citrate.

8. The method of claim 1, wherein the antiestrogen is administered orally.

9. The method of claim 1, wherein the human male is administered an aromatase inhibitor and is not administered an antiestrogen.

10. The method of claim 3, wherein the aromatase inhibitor is selected from anastrozole, letrozole, exemestane, vorozole, formestane, fadrozole, aminoglutethimide, testolactone, 4-hydroxyandrostenedione, 1,4,6-androstatrien-3, 17-dione and 4-androstene-3,6, 17-trione.

11. The method of claim 3, wherein said co-administration comprises a period where administration of the antiestrogen and administration of the aromatase inhibitor overlap.

12. A method for reversing secondary hypogonadism in a human male with secondary hypogonadism and a BMI ≥30 (e.g. BMI ≥35) who is less than 60 years of age by administering an effective amount of an antiestrogen and/or aromatase inhibitor for a period of at least about six months in combination with a prescribed weight loss regimen, whereby LH and testosterone levels in the subject remain in the normal range for a period of at least 6 months after cessation of treatment with the antiestrogen and/or aromatase inhibitor.

13. The method of claim 12, wherein the human male is administered an effective amount of a pharmaceutical composition comprising trans-clomiphene, the composition being substantially free of cis-clomiphene.

14. The method of claim 1, wherein the human male
(a) has a body mass index ≥35, and/or
(b) has a body mass index between 35 and 45 and/or
(c) has a testosterone:estrogen (T:E) ratio of ratio of below about 10, and/or
(d) has a T:E ratio of below about 7 and/or
(e) has a T:E ratio below about 5 and/or
(f) has a T:E ratio below about 3 and/or
(g) has an estradiol level between 30 and 60 μg/ml and/or
(h) has an estradiol level between 40 and 60 μg/ml and/or
(i) has an estradiol level between 45 and 55 μg/ml.
